(19) United States
(12) Patent Application Publication
(10) Pub. No.: US 2025/0144355 A1
(43) Pub. Date: May 8, 2025

(54) APPARATUS AND PROCESS FOR ANESTHETIZING A PATIENT

(71) Applicant: Drägerwerk AG & Co. KGaA, Lübeck (DE)

(72) Inventors: Ulf PILZ, Lübeck (DE); Andreas KATER, Lübeck (DE)

(21) Appl. No.: 18/938,527

(22) Filed: Nov. 6, 2024

(30) Foreign Application Priority Data
Nov. 7, 2023 (DE) 10 2023 130 720.7

(51) Int. Cl. A61M 16/10; A61M 16/00; A61M 16/20

(52) U.S. Cl. CPC A61M 16/104; A61M 16/022; A61M 16/1005; A61M 16/208

(57) ABSTRACT

A ventilation arrangement (200) and process ventilate a patient (P) with a ventilation circuit (40) connecting a ventilator (1) to a patient-side coupling unit (21). The ventilator performs a sequence of ventilation strokes, including expelling a quantity of a gas mixture that flows in the ventilation circuit to the patient-side coupling unit. Gas mixture exhaled by the patient flows in the ventilation circuit from the patient-side coupling unit to the ventilator. A gas mixture supply unit (31, 48) expels an additional gas mixture into the ventilation circuit at a feed point (38). A specification sets a volume flow of the additional gas mixture. In response to capturing a specification, a propagation duration for the expelled additional gas mixture to reach the feed point is predicted. The additional gas mixture is controlled to ensure that the expelled additional gas mixture reaches the feed point in an expiration phase.

### CROSS REFERENCE TO RELATED APPLICATIONS

This application claims the benefit of priority under 35 U.S.C. § 119 of German Application 10 2023 130 720.7, filed Nov. 7, 2023, the entire contents of which are incorporated herein by reference.

### TECHNICAL FIELD

The present invention pertains to an arrangement and a process for artificially ventilating (artificially respirating) a patient and thereby anesthetizing him/her.

### BACKGROUND

In order to anesthetize a patient and to ventilate the anesthetized patient artificially, usually a ventilation circuit is established between a ventilator and a patient-side coupling unit. The patient-side coupling unit is positioned in and/or on the patient's body. The ventilator expels (emits/ejects) a breathable gas mixture comprising oxygen and an anesthetic agent, usually in the form of ventilation strokes. This breathable gas mixture is fed (guided) to the patient-side coupling unit in the ventilation circuit. A gas mixture that the patient has exhaled is guided back to the ventilator in the ventilation circuit. The exhaled gas mixture usually contains exhaled anesthetic. The invention can be used in such a ventilation circuit.

### SUMMARY

It is an object of the invention to provide a ventilation arrangement and a ventilation process for artificial ventilation of a patient which, in certain situations, ensure greater operational safety than known ventilation arrangements and ventilation processes.

The object is attained by a ventilation arrangement with ventilation arrangement features according to the invention and by a ventilation process with ventilation process features according to the invention. Advantageous embodiments of the ventilation arrangement according to the invention are, where appropriate, also advantageous embodiments of the ventilation process according to the invention and vice versa.

The ventilation arrangement according to the invention and the ventilation process according to the invention are configured to artificially ventilate a patient. During artificial ventilation, the patient is permanently or at least temporarily connected to a patient-side coupling unit. The patient-side coupling unit is arranged in and/or on the patient's body. A breathing mask on the patient's face and a tube in the patient's air tube (windpipe) are two examples of a patient-side coupling unit. It is possible that the patient is completely anesthetized and is only ventilated by a ventilator. It is also possible that the patient performs his/her own respiratory activity during artificial ventilation, for example due to his/her spontaneous breathing and/or because his/her respiratory muscles are stimulated externally.

A ventilator of the ventilation arrangement is configured to perform a sequence of ventilation strokes. In each ventilation stroke, the ventilator is configured to expel a respective quantity of a breathable gas mixture. This breathable gas mixture comprises oxygen and optionally at least one anesthetic and/or at least one drug. It is possible that the proportion (concentration, share) of the or at least one anesthetic agent in the expelled breathable gas mixture is changed during the artificial ventilation of a patient, for example due to a corresponding specification (setting/preset value) given by a user. It is also possible that the drug is only added to the breathable gas mixture during artificial ventilation.

The ventilation arrangement provides a ventilation circuit. The ventilation circuit connects the ventilator to the patient-side coupling unit and is located partly in the ventilator and partly outside the ventilator. The ventilator arrangement is configured to guide, in particular to convey, the respective amount of breathable gas mixture expelled in a ventilation stroke in the ventilation circuit to the patient-side coupling unit. The patient is configured to inhale this conveyed quantity of breathable gas mixture.

The patient exhales a quantity of a gas mixture. As a rule, this exhaled quantity contains carbon dioxide and at least one anesthetic. The ventilation arrangement is configured to guide the exhaled quantity from the patient-side coupling unit in the ventilation circuit to the ventilator, in particular to convey it.

A gas mixture supply unit is configured to expel an additional gas mixture. As a rule, the additional gas mixture comprises oxygen and preferably at least one anesthetic agent and/or a gaseous drug. The additional gas mixture may consist of pure oxygen. The ventilation arrangement is configured to convey and guide the expelled additional gas mixture to a feed point and to feed the expelled additional gas mixture into the ventilation circuit at the feed point.

The gas mixture supply unit is configured to expel the additional gas mixture with a variable actual volume flow (volume stream). The volume flow is the volume per unit of time of the additional gas mixture that leaves the gas mixture supply unit. As a rule, a target volume flow is specified, in particular based on a specification (setting) from a user or a higher-level control system. It is also possible that the specification specifies a target mass flow instead of or in addition to the target volume flow. The mass flow is the mass per unit time of the additional gas mixture. The term “volume flow” as used below also includes the mass flow.

The ventilation process according to the invention is carried out using such a ventilation arrangement, preferably while the patient is connected to the patient-side coupling unit and the ventilation circuit is established.

A signal-processing control unit is configured to capture (record/acquire/detect) a specification. The specification comes, for example, from a user or from a higher-level control system. The specification specifies a target volume flow. The gas mixture supply unit should expel the additional gas mixture with this specified and captured target volume flow. In addition, the control unit is configured to control the gas mixture supply unit and cause the controlled gas mixture supply unit to expel the additional gas mixture in accordance with the captured specification.

The control unit is configured to perform the following steps automatically and in response to capturing the specification, namely before the control unit triggers the execution (implementation) of the specification by activating the gas mixture supply unit:

A propagation duration is automatically predicted. This propagation duration elapses between the following two events:
The gas mixture supply unit expels the additional gas mixture in accordance with the captured specification.
The expelled additional gas mixture reaches the feed point.

The propagation duration is predicted as a function of the target volume flow, which is specified in the captured specification, and optionally of other parameters. The optional uses of further parameters represent advantageous embodiments of the invention.

The gas mixture supply unit is controlled (activated). The objective of the control is to ensure that the controlled gas mixture supply unit expels the additional gas mixture in accordance with the captured specification.

Controlling the gas mixture supply unit has the following effect: The additional gas mixture expelled according to the captured specification reaches the feed point within an expiration phase (expiratory phase) or within an optional intermediate phase, but not within an inspiration phase (inspiratory phase). In other words, the time span in which the additional gas mixture reaches the feed point does not overlap with an inspiration phase. This is achieved by controlling the gas mixture feed unit accordingly. The control unit uses the predicted propagation duration and optionally other parameters to achieve through suitable control this desired effect.

It is therefore possible that the captured specification is not executed immediately, but with a time delay. In other words, if required, the process of the gas mixture supply unit expelling the additional gas mixture is not triggered immediately after the specification is captured, but with a sufficiently long time delay. Preferably, this time delay is as short as possible and only as long as necessary. As a result, the captured specification is executed (implemented) as quickly as possible, but without endangering the patient.

Note: It is possible that the gas mixture supply unit expels the additional gas mixture before the specification is captured, but usually in such a way that the actual volume flow is less than the target volume flow specified in the specification. It is also possible that the additional feed unit only expels the additional gas mixture after the specification has been captured, i.e. not before.

An expiration phase is a period during which a gas mixture exits the patient-side coupling unit to flow to the ventilator. This gas mixture was or will be exhaled by the patient into the patient-side coupling unit. An inspiration phase, on the other hand, is a phase in which a breathable gas mixture flows into the patient-side coupling unit and can be inhaled by the patient. As the ventilator performs the ventilatory strokes, the ventilator causes the inspiration phases, namely one inspiration phase per ventilatory breath. As a rule, the expiration and inspiration phases do not overlap.

Optionally an intermediate phase occurs between an inspiration phase and an expiration phase. In this optional intermediate phase, neither a breathable gas mixture expelled in a ventilation stroke and having a volume flow above an upper threshold flows to the patient-side coupling unit nor an exhaled gas mixture with a volume flow above this upper threshold flows away from the patient-side coupling unit. It is also possible that an inspiration phase is immediately followed by an expiration phase and/or, conversely, an expiration phase is immediately followed by an inspiration phase.

Note: It is possible that, at least temporarily, an auxiliary gas mixture flows in the ventilation circuit that is not delivered due to a ventilation stroke. For example, a fluid guide unit of the ventilation circuit is flushed out, or a gas sample is extracted from the ventilation circuit, analyzed and fed back into the ventilation circuit. These gas mixtures do not contribute to the expiration and inspiration phases. However, they can contribute to mixing the additional gas mixture fed in with a gas mixture already present in the ventilation circuit.

The ventilation process comprises the corresponding steps that are carried out after the specification regarding the desired target volume flow from the gas mixture supply unit has been captured.

According to the invention, a ventilation circuit is provided. In this ventilation circuit, an expelled breathable gas mixture comprising oxygen flows from the ventilator to the patient-side coupling unit, and conversely, an exhaled gas mixture flows from the patient-side coupling unit to the ventilator. If the breathable gas mixture which the ventilator expels and which flows to the patient-side coupling unit contains an anesthetic agent or a gaseous drug, the exhaled gas mixture usually also contains the anesthetic agent and/or the gaseous drug. Because a ventilation circuit is provided, there is a relatively low risk of anesthetic or drug escaping into an area surrounding the ventilation arrangement. This is undesirable. In addition, in many cases at least part of the exhaled anesthetic can be reused if it remains in the ventilation circuit or is diverted from the ventilation circuit and reprocessed.

According to the invention, the control unit captures a specification, in particular a preset value or a set value, wherein the captured specification specifies a target volume flow of the additional gas mixture. The invention reduces the risk of the artificially ventilated patient being endangered by the execution of the captured specification. This risk exists, in particular, if the specification specifies that the target volume flow should be increased relatively quickly and/or relatively strongly. If this specification would in any case be implemented immediately and no countermeasure is taken, the following risk exists: A quantity of the additional gas mixture flows with a relatively large actual volume flow to the feed point, there into the ventilation circuit and further in the ventilation circuit to the patient-side coupling unit. The patient then temporarily inhales a gas mixture with a relatively large amount of the additional gas mixture. The inhaled additional gas mixture contains, for example, at least one anesthetic and/or a drug and/or pure oxygen. If the patient inhales a large quantity of the anesthetic agent and/or the drug and/or pure oxygen practically abruptly, this means a bolus of this substance. This bolus can endanger the patient and is therefore undesirable in many cases. A sudden supply of large amounts of oxygen could also endanger the patient in some situations.

Note: The gas mixture supply unit often generates the additional gas mixture from several gas components, whereby one gas component is an anesthetic, for example. In response to the specification regarding the target volume flow of the additional gas mixture having been captured, the gas mixture supply unit often reduces the proportion of a gas component in the additional gas mixture. However, this reduction often only takes effect with a time delay. A quantity of the additional gas mixture that the gas mixture supply unit has already expelled still has the old proportion of the gas component, for example the old proportion of anesthetic or a drug. A surge with the old portion of the gas component often reaches the ventilation circuit and later the patient-side coupling unit, which could cause a bolus if no countermeasure is performed.

According to the invention, the risk of the patient being endangered by a bolus of the additional gas mixture is reduced by ensuring the following: The additional gas mixture expelled according to the specification reaches the feed point in an expiration phase or in an optional intermediate phase, but not in an inspiration phase. In an expiration phase or in an intermediate phase, the patient generally does not inhale any gas mixture and therefore does not inhale the injected additional gas mixture.

In the expiration phase or in the intermediate phase, the injected additional gas mixture mixes with a gas mixture that is already in the ventilation circuit. The patient only inhales a gas mixture again in a later phase, e.g. in the immediately following inspiration phase. Therefore, the inhaled gas mixture usually consists of a mixture of the gas mixture that was already present in the ventilation circuit before the injection with the injected additional gas mixture. In the situation just described, in which the additional gas mixture contains an anesthetic and/or a drug and/or pure oxygen and the specification specifies an increase in the target volume flow, the invention leads to the following: The actual volume flow of anesthetic agent and/or the drug and/or pure oxygen is lower in a subsequent inspiration phase than if, in the inspiration phase, the injected additional gas mixture had been fed to the patient-side coupling unit without dilution, i.e. without the injected additional gas mixture having been diluted by the gas mixture already present in the ventilation circuit.

According to the invention, it is ensured that the expelled additional gas mixture reaches the feed point in an expiration phase or in an optional intermediate phase. As a result, a sufficiently long time period generally elapses for mixing until a gas mixture with the injected additional gas mixture reaches the patient-side coupling unit in a subsequent inspiration phase. This effect is achieved regardless of the flow rate in the ventilation circuit and also regardless of the length of a section in the ventilation circuit between the feed point and the patient-side coupling unit. Therefore, the two parameters flow velocity and length of the circuit do not necessarily need to be measured or specified.

The invention reduces the risk of the patient's health being endangered, namely by a bolus. This effect is achieved without the need of changing or amending or adapting the sequence of ventilation strokes. It is often desirable for the sequence of ventilation strokes to be well synchronized with the patient's own respiratory activity. In particular for this reason, it is usually desirable not to have to change the sequence of ventilation strokes due to the introduction of the additional gas mixture.

As already explained, the introduction of the additional gas mixture can lead to a bolus and thus to a risk to the patient's health if this additional gas mixture reaches the patient-side coupling unit as quick as technically possible. One conceivable countermeasure to avoid this undesirable event is the following: At least part of the injected additional gas mixture is removed from the ventilation circuit again before it reaches the patient-side coupling unit. In many cases, however, this leads to a waste of anesthetic or drug. The invention thus presents a way to reduce the waste of anesthetic or a drug.

The invention makes it possible for the actual volume flow with which the additional gas mixture is fed in to be increased abruptly and/or greatly, thereby also increasing an actual volume flow of anesthetic agent and/or a drug or other substance, without endangering the patient's health. It is possible, but thanks to the invention not necessary, to limit the achievable increase in the volume flow of the additional gas mixture.

According to the invention, the gas mixture supply unit is configured to expel the additional gas mixture. Thanks to the invention, it is possible for the gas mixture supply unit to expel the additional gas mixture continuously and therefore also generate it continuously. It is also possible for the additional gas mixture to be expelled in an oscillating manner, whereby the oscillations may be independent of the ventilation strokes. Thanks to the invention, it is not necessary to adapt the process of expelling the additional gas mixture to the inspiration phases and/or to the expiration phases and/or to the ventilation strokes. This adaptation would require, for example, generating the additional gas mixture only in some phases or generating it continuously but storing it temporarily. Continuous operation without intermediate storage is generally easier to implement and is more reliable than an operation that must be adapted to the inspiration phases and/or the expiration phases and/or to the ventilation strokes.

As a rule, the invention can be implemented purely by modifying the software on the control unit accordingly. It is often not necessary to change the hardware of the ventilator or any other component of the ventilator circuit. In particular, it is usually not necessary to add a sensor in order to realize the invention. The parameters required to predict the propagation duration result from the captured specification and/or from the design and construction of the ventilator arrangement, optionally from a physical variable for which a sensor is usually already present. The fact that the invention can in many cases be implemented exclusively by modified software makes it easier to integrate the invention into an existing ventilation arrangement.

The invention ensures that the additional gas mixture reaches the feed point within an expiration phase or within an optional intermediate phase, but not overlapping with an inspiration phase. In one embodiment, this feature is ensured for every additional gas mixture. In another embodiment, it depends on the additional gas mixture whether it is ensured that the additional gas mixture only reaches the feed point in an expiration phase or an optional intermediate phase. For example, this is ensured if the additional gas mixture comprises an anesthetic and/or a drug. If, on the other hand, the additional gas mixture consists of pure oxygen, the pure oxygen is fed to the feed point independently of the inspiration and expiration phases, i.e. without a time delay depending on the propagation duration. One reason for this is that the injection of pure oxygen is generally less dangerous for the patient than a bolus with an anesthetic or drug. In addition, in some situations the injection of pure oxygen has high priority and/or urgency.

According to the invention, the ventilation arrangement is configured to guide the expelled additional gas mixture from the gas mixture supply unit to the feed point. Preferably, a fluid guide unit is used for this purpose, which fluid guide unit leads from the gas mixture supply unit to the feed point. The expelled additional gas mixture thus flows from the gas mixture feed unit through this fluid guide unit to the feed point. According to the invention, the specified and captured target volume flow is used to predict the propagation duration. Preferably, a predetermined volume of the fluid guide unit, which leads from the gas mixture feed unit to the feed point, is also used for this prediction. This volume is known from the design of the ventilation arrangement and usually remains unchanged during use. Optionally, a pneumatic resistance of the fluid guide unit is also used. This resistance is also known from the design.

Notes:
A fluid guide unit is a component that is configured to guide a fluid along a trajectory, whereby this trajectory is predetermined by the configuration and/or the arrangement of the component. Ideally, the fluid guide unit prevents the fluid from leaving the trajectory. A hose and a tube are two examples of a fluid guide unit. A fluid conveying unit (fluid delivery unit) is configured to convey a fluid along a fluid guide unit. A blower (fan), a pump, and a piston-cylinder unit are examples of a fluid conveying unit.
The pneumatic resistance of a fluid guide unit is understood to be the quotient of the pressure drop (pressure loss) at this fluid guide unit and the volume flow through the fluid guide unit. Often the assumption is justified that the pneumatic resistance does not depend on the volume flow.

According to the invention, the additional gas mixture, which is expelled by the controlled gas mixture supply unit, reaches the feed point and thus the ventilation circuit in an expiration phase or in an optional intermediate phase. In one embodiment, a mixing duration (mixing time) is specified. At least this mixing duration is required for the step that the additional gas mixture, which is fed into the ventilation circuit, can mix sufficiently well with a gas mixture that is already present in the ventilation circuit before the additional gas mixture is fed in. As a rule, this already present gas mixture comprises gas that the patient has exhaled. Mixing prevents the undesired event that one component of the additional gas mixture has locally a very high concentration. The control unit is preferably configured to control the gas mixture supply unit as follows: The expelled additional gas mixture reaches the feed point in an expiration phase or in an intermediate phase in such a way that the following is ensured: At least the predetermined mixing duration elapses between the event that the additional gas mixture reaches the feed point and the start of the next inspiration phase or at least the end of this expiration phase.

This design reduces the risk of the following undesirable event occurring: The expelled additional gas mixture reaches the feed point in an expiration phase, but neither in this expiration phase nor in an optional subsequent intermediate phase enough time is available for the injected additional gas mixture to mix sufficiently well with a gas mixture that is already present in the ventilation circuit. Insufficient or even no mixing can lead to a bolus.

This embodiment provides for the following: If required, the time at which the expelling of the additional gas mixture is triggered is delayed. If required, the control unit delays the activation of the gas mixture feed unit. Preferably the activation is delayed such that the expelled additional gas mixture reaches the feed point in the next expiration phase or in a subsequent intermediate phase.

According to the invention, the ventilation circuit provided connects the ventilator to the patient-side coupling unit. In one embodiment, the ventilation arrangement comprises a valve. This valve is arranged in the ventilation circuit and there between the feed point and the patient-side coupling unit. The valve can either open or close the ventilation circuit. The ventilation arrangement is configured in such a way that the following two effects are achieved:
During a ventilation stroke and therefore during the inspiration phase, the valve releases a segment in the ventilation circuit. This segment extends between the feed point and the patient-side coupling unit.
In an expiration phase and preferably also in an intermediate phase, the valve blocks this segment.

In accordance with the invention, the invention ensures that the expelled additional gas mixture is only fed into the ventilation circuit in an expiration phase or in an optional intermediate phase. The valve then blocks the segment. The valve thus further reduces the risk of the undesired event of the injected additional gas mixture flowing directly to the patient-side coupling unit, i.e. without first mixing with a gas mixture that is already present in the ventilation circuit. In particular, the valve can be configured as a purely mechanical non-return valve or as a controllable non-return valve or proportional valve.

According to the invention, the gas mixture supply unit is configured to generate and expel an additional gas mixture. Preferably, the gas mixture supply unit comprises two inlets and one outlet. At one inlet, the gas mixture supply unit is supplied with an active substance, preferably an active substance in liquid form, for example an anesthetic or a drug. The gas mixture supply unit is supplied with a carrier gas at the other inlet. At the outlet, the gas mixture supply unit provides a gas mixture of the active ingredient in gaseous form and the carrier gas.

In one embodiment, the gas mixture supply unit comprises an anesthetic vaporizer (anesthetic evaporator/anesthetic dispenser). The anesthetic vaporizer preferably has an inlet for a carrier gas and an inlet for a liquid anesthetic. Thanks to the anesthetic vaporizer, the gas mixture supply unit is configured to generate and emit an additional gas mixture which has the following property: The additional gas mixture comprises a carrier gas and at least one gaseous anesthetic agent, optionally several gaseous anesthetic agents. The carrier gas comprises pure oxygen (O₂) and/or breathing air and, in one embodiment, additionally nitrous oxide (N₂O).

Preferably, the anesthetic vaporizer comprises an evaporation chamber. The carrier gas flows through this evaporation chamber. In the evaporation chamber, the anesthetic vaporizer is configured to feed an anesthetic into a flow of carrier gas, in particular by evaporation or vaporization or also by injection. The anesthetic vaporizer can therefore be configured as a vaporizer.

In one embodiment, the anesthetic vaporizer comprises a vaporizer module and/or an injection module as well as a mixing chamber. The vaporizer module receives liquid anesthetic from a storage container and vaporizes or evaporates it. The injection module receives the liquid anesthetic and injects it into the carrier gas. Preferably, the liquid anesthetic is heated in a further chamber of the vaporizer module. In the mixing chamber, the gaseous anesthetic is mixed with a stream of carrier gas, in particular by injection. In this case, the mixing chamber acts as the evaporation chamber.

According to the invention, the control unit is configured to predict a propagation duration. The propagation duration is the time span between the event that the additional gas mixture is expelled and the event that the expelled additional gas mixture reaches the feed point. According to the embodiment just described, the control unit additionally predicts the propagation duration depending on a predetermined volume of the evaporation chamber. This embodiment takes into account the fact that the actual volume flow of the additional gas mixture in this application is often essentially determined by the actual volume flow of the carrier gas, but only little by the actual volume flow of the anesthetic. Therefore, the propagation duration additionally depends on the volume of the evaporation chamber. As a rule, the volume of the evaporation chamber has a much smaller influence on the propagation duration than the specified target volume flow and the volume of a fluid guide unit from the anesthetic vaporizer to the feed point.

According to the invention, the control unit is configured to capture a specification, whereby the captured specification specifies a target volume flow. The gas mixture supply unit is intended to expel the additional gas mixture at this specified target volume flow, i.e. to actually achieve the target volume flow. In one embodiment, the gas mixture supply unit already expels the additional gas mixture when the control unit captures the specification, but in such a way that the previous actual volume flow is less than the target volume flow specified in the specification. Preferably, the target volume flow according to the specification is at least 50% greater than, and particularly preferably at least twice as great as, the current actual volume flow in the moment at which the specification is captured and before the specification is executed (implemented). The specification therefore specifies a sharp increase in the volume flow. If this specification is actually executed and the actual volume flow is suddenly increased, a surge of the additional gas mixture flows to the feed point. The invention causes this surge to be injected in an expiration phase or in an optional intermediate phase, and also reduces in this situation the risk that the patient inhales a too large quantity of the additional gas mixture, i.e. the risk that an undesired bolus occurs.

The invention is described below by means of embodiment examples. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a ventilation arrangement with a ventilation circuit for artificial ventilation of a patient during inspiration;

FIG. 2 is a schematic view of the ventilation arrangement with the ventilation circuit of FIG. 1, shown during an expiration phase;

FIG. 3 is a view showing a first exemplary time sequence in which the captured specification is immediately executed;

FIG. 4 is a view showing a second exemplary time sequence in which the captured specification is executed with a delay so that the surge is fed in during an expiration phase;

FIG. 5 is a view showing a third exemplary time sequence in which the captured specification is executed with a delay so that a flow surge can mix well.

### DESCRIPTION OF PREFERRED EMBODIMENTS

Referring to the drawings, in the embodiment examples, the invention is used for the artificial ventilation of a patient. The patient is supplied with a breathable gas mixture comprising oxygen and at least one anesthetic agent. The patient is therefore anesthetized or at least sedated.

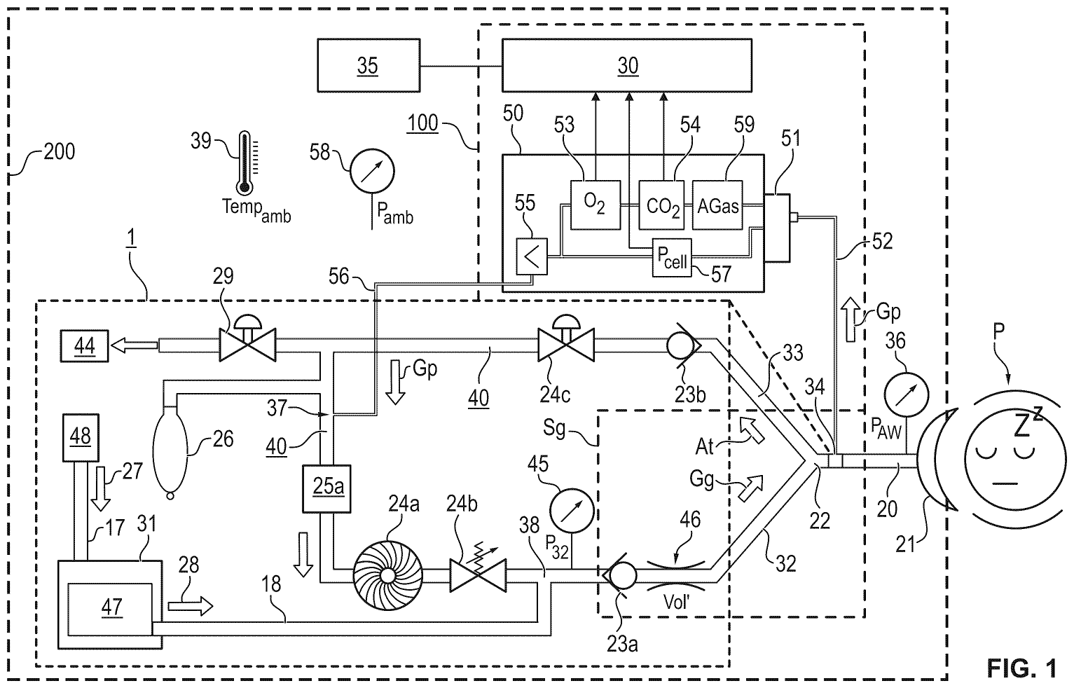

FIG. 1 schematically shows a ventilation arrangement 200 with a schematically shown ventilator in the form of an anesthesia machine 1, whereby the ventilation arrangement 200 is configured to artificially ventilate a patient P. Only the face of patient P is shown schematically. The anesthesia machine 1 maintains a gas flow in a ventilation circuit 40. The flow direction in the ventilation circuit 40 is indicated by arrows.

A patient-side coupling unit 21, shown only schematically, for example a mouthpiece or a breathing mask or a tube, is arranged in and/or on the body of the patient P and connects the patient P to the ventilation circuit 40. The patient-side coupling unit 21 is connected to the base part of a Y-piece 22 via a tube 20, in the embodiment example a corrugated tube 20. One leg of the Y-piece 22 is connected to an inhalation breathing gas line 32 for inhalation (inspiration), the other leg is connected to an exhalation breathing gas line 33 for exhalation (expiration). Both breathing gas lines 32, 33 belong to the ventilation circuit 40.

In the ventilation circuit 40, a breathable gas mixture Gg is supplied to the patient P. In the embodiment example, this breathable gas mixture Gg comprises oxygen (O₂), nitrogen (N₂), at least one anesthetic agent and optionally a carrier gas for an anesthetic agent, e.g. nitrous oxide (N₂O).

To supply the breathable gas mixture Gg to the patient P, the anesthesia machine 1 performs a sequence of ventilation strokes. In each ventilation stroke, the anesthesia machine 1 expels a respective quantity of the breathable gas mixture Gg. The expelled breathable gas mixture Gg is fed to the patient-side coupling unit 21 in the ventilation circuit 40. The gas mixture At, which is exhaled by the patient P into the patient-side coupling unit 21 or otherwise leaves the body of the patient P, contains anesthetics and carbon dioxide and flows back in the ventilation circuit 40 to the anesthesia machine 1.

It is possible that the patient P is not completely anesthetized, but performs his/her own respiratory activity. Patient P's own respiratory activity is caused by his/her spontaneous breathing and optionally by external stimulation of his/her respiratory muscles. Ideally, in this application, the ventilatory strokes of the anesthesia machine 1 are precisely synchronized with patient P's own respiratory activity. As a rule, this ideal synchronization can only be achieved approximately.

The sequence of ventilation strokes results in a sequence of inspiration phases and a sequence of expiration phases. In each inspiration phase, the quantity of the breathable gas mixture Gg expelled in a corresponding ventilation stroke reaches the patient-side coupling unit 21. In each expiration phase, the patient P exhales a quantity of the gas mixture At into the patient-side coupling unit 21. The inspiration phases and the expiration phases do not overlap in time. In one embodiment, an intermediate phase occurs between an inspiration phase and a subsequent expiration phase, in which a gas mixture with a volume flow above a predetermined lower limit flows neither towards the patient-side coupling unit 21 nor away from the patient-side coupling unit 21. As a rule, artificial ventilation consists of the following phases: Inspiration phase-intermediate phase-expiration phase-intermediate phase-inspiration phase, etc.

An anesthetic vaporizer 31 generates a fluid flow 28 with vaporous anesthetic. The fluid flow 28 with the anesthetic component is guided through a fluid guide unit 18 and fed into the ventilation circuit 40 at a feed point 38. The gas mixture in the ventilation circuit 40 receives the fed in additional gas mixture.

The fluid flow 28 is generated as follows: A fluid flow 27 with a carrier gas flows through a fluid guide unit 17 to an evaporation chamber 47 of the anesthetic vaporizer 31. To generate the fluid flow 28, the anesthetic vaporizer 31 evaporates anesthetic and feeds it into the fluid flow 27 in the evaporation chamber 47. The anesthetic agent fed in comes, for example, from a tank with liquid anesthetic agent (not shown) or from a conditioner (purifier) 44, whereby the conditioner 44 processes an exhaled gas mixture At and extracts the anesthetic agent from it. An evaporator module of the anesthetic vaporizer 31 evaporates the liquid anesthetic and the gaseous anesthetic is fed into the fluid flow 27 in the evaporation chamber 47.

The carrier gas in the fluid flow 27 comprises breathing air and/or pure oxygen and optionally nitrous oxide (N₂O) and is generated by a mixer 48. The mixer 48 receives the components of the carrier gas from, for example, three supply connections or from, for example, three storage containers. Preferably, the mixer 48 is controlled with the control objective (control gain) that the volume flow of the carrier gas actually generated by the mixer 48 is equal to a predetermined target volume flow. The volume flow with which the gas mixture comprising anesthetic leaves the anesthetic vaporizer 31 essentially corresponds to the volume flow with which the mixer 48 actually expels the carrier gas. It is possible that the mixer 48 is additionally controlled with the further control objective that the actual chemical composition of the carrier gas is equal to a predetermined target composition.

At least as long as there is no excessive overpressure in the ventilation circuit 40, the gas mixture At exhaled by the patient P remains in the ventilation circuit 40, so that no anesthetic or drug is released into the environment by exhalation. An overpressure is preferably reduced in such a way that no anesthetic is released into the environment, but is releases into the conditioner 44, e.g., even when the overpressure is reduced.

Inevitably, the gas mixture At, which flows away from the patient P through the ventilation circuit 40, contains carbon dioxide (CO₂) exhaled by the patient P. A carbon dioxide absorber (CO₂ absorber) 25a is configured to absorb carbon dioxide from the ventilation circuit 40. In addition, the gas mixture Gg, At in the ventilation circuit 40 may contain other components that also occur in the ambient air, but possibly at a different concentration than in the ambient air.

A fluid conveying unit 24a generates a flow of a breathable gas mixture Gg in the ventilation circuit 40. The flow of a gas mixture in the ventilation circuit 40 is kept going by the fluid conveying unit 24a and optionally by a manual ventilation bag 26, which can be operated manually. In the embodiment example, the fluid conveying unit 24a has the form of a blower, which constantly generates a fluid flow. The use of a blower makes it easier to synchronize the ventilation strokes with the patient P's own respiratory activity. It is also possible that the fluid conveying unit 24a comprises a pump, a bellows or a piston-cylinder unit.

In the embodiment example, the fluid conveying unit 24a continuously delivers (conveys) a breathable gas mixture Gg. Preferably, the fluid conveying unit 24a is controlled with the control objective (control gain) that the pressure at the outlet of the fluid conveying unit 24a remains constant, for example a constant 5 mbar. Depending on its position, a controllable proportional valve 24b allows the fluid flow generated by the fluid conveying unit 24a to pass or blocks it fully or at least partially, thereby contributing to the generation of the individual ventilation strokes and determining the respective start, the respective end, the amplitudes and frequencies of these ventilation strokes. In each ventilation stroke, a respective quantity of the breathable gas mixture Gg is fed through the inhalation-breathing gas line 32 to the Y-piece 22 and to the patient-side coupling unit 21 and thus to the patient P. In the embodiment example, the controlled valve 24b is located between the fluid conveying unit 24a and the feed point 38. It is also possible that the feed point 38 is located between the fluid conveying unit 24a and the valve 24b.

A PEEP valve 24c (PEEP=“positive end-expiration pressure”) ensures that there is always sufficient pressure in the lungs of patient P, thereby preventing the lungs from collapsing and/or becoming collapsed. This effect is also ensured at the end of an exhalation and when the ventilation circuit 40 is briefly opened or interrupted.

A non-return valve (check valve) 23a allows a gas flow to flow through the inhalation breathing gas line 32 towards the Y-piece 22 and blocks a gas flow in the opposite direction. Depending on its position, the non-return valve 23a thus releases or blocks a segment Sg of the ventilation circuit 40. This segment Sg extends between the feed point 38 and the patient-side coupling unit 21. A non-return valve 23b allows a gas flow through the exhalation breathing gas line 33 away from the Y-piece 22 and blocks a gas flow in the opposite direction. In the embodiment example, the two non-return valves 23a and 23b are configured as purely mechanical valves and open or close depending on which side has the greater pressure.

A pressure relief valve 29 is configured to reduce excess pressure in the ventilation circuit 40 by causing gas to escape from the ventilation circuit 40 if the pressure is too high. Preferably, the released gas is discharged into a transport line for anesthetic gas and reaches a conditioner 44, as shown schematically. This feature prevents the anesthetic agent from escaping into the environment even in the event of excess pressure. In addition, the consumption of anesthetic is reduced.

A signal-processing ventilation control unit 35, shown only schematically, receives a signal from an optional pressure sensor 58, which measures the air pressure P_{amb} in the environment of the anesthesia machine 1 and thus in the environment of the ventilation circuit 40, and a signal from an optional temperature sensor 39, which measures the ambient temperature Temp_{amb}. The ventilation control unit 35 also receives
a signal from a pressure sensor 45, which measures the time-varying pressure P₃₂ at a measuring position in the ventilation circuit 40, in this case the pressure in the inhalation breathing gas line 32, and
a signal from a pressure sensor 36, which measures the ventilation pressure, whereby the measured ventilation pressure is applied to the patient-side coupling unit 21 and determines the airway pressure (pressure in airway, Paw) at the patient P.

In one embodiment, the pressure sensors 45 and 36 measure the differential pressure relative to the ambient pressure P_{amb}. A volume flow sensor 46 measures the volume flow (volume stream) Vol′, i.e. the volume per unit time, through the inhalation breathing gas line 32, in the embodiment example at a measuring point downstream of the feed point 48 and downstream of the non-return valve 23a. The ventilation control unit 35 receives a respective signal from the volume flow sensor 46, the pressure sensors 45 and 36 and from the optional temperature sensor 39. A volume flow sensor (not shown) measures the volume flow through the exhalation breathing gas line 33.

Note: The phrase that a sensor measures a physical quantity is used repeatedly. This phrase means that the sensor directly measures this physical quantity or another quantity that correlates with the physical quantity to be measured. The other quantity is therefore an indicator of the quantity to be measured.

Viewed in the direction of flow of a gas mixture through the ventilation circuit 40, the components just mentioned are arranged in the following order (sequence) in the embodiment example, see FIG. 1:
the carbon dioxide absorber 25a,
the fluid conveying unit 24a,
the controllable proportional valve 24b,
the feed point 38,
the measuring position for the pressure sensor 45,
the non-return valve 23a, and
the measuring position for the volume flow sensor 46.

A different arrangement is also possible.

In order to achieve the desired artificial ventilation and anesthesia of the patient P, the ventilation control unit 35 controls the fluid conveying unit 24a, the valve 24b, optionally the PEEP valve 24c, the anesthetic vaporizer 31, the mixer 48 and other components of the ventilation circuit 40. For this control, the ventilation control unit 35 processes signals from the sensors 36, 46, 39, 58 as well as captured specifications (settings) from a user and/or a higher-level control system.

In one embodiment, the ventilation control unit 35 performs a closed-loop control. One control objective is that the actual time course of the volume flow Vol′ through the inhalation breathing gas line 32 and/or the actual time course of the pressure Paw at the patient-side coupling unit 21 follows a predetermined time course. The first alternative is often referred to as volume-controlled control, the second alternative as pressure-controlled control. For this control, the ventilation control unit 35 processes signals from the sensor 46 and/or the sensor 35. In order to change the actual volume flow or pressure, the ventilation control unit 35 controls in particular the valve 24b.

It is often desired that the breathable gas mixture Gg, which is supplied to the patient P, fulfills a certain property. In particular, the concentration (proportion) of a component of the breathable gas mixture Gg should lie within a predetermined target range. For example, the proportion of anesthetic should often be within a predetermined range. On the one hand, the proportion of anesthetic agent should be large enough to ensure that patient P is reliably anesthetized. On the other hand, patient P must not be endangered by a too high proportion of anesthetic. In the following, the term “concentration” of a gas component in a gas mixture is used. A synonymous term is “proportion” (or share).

The ventilation control unit 35 preferably controls the anesthetic vaporizer 31 with the control objective of ensuring that the actual proportion of anesthetic in the fluid flow 28 is equal to a predetermined proportion of anesthetic. This predetermined proportion of anesthetic can be constant over time or vary over time.

The above-mentioned controls require that the actual respective proportion of various components in the breathable gas mixture Gg is measured. For this purpose, a sample (hereinafter: a gas sample Gp) of the breathable gas mixture Gg is taken (branched off) from the ventilation circuit 40 via a sampling tube 52, analyzed and fed back into the ventilation circuit 40 via a discharge tube 56. The sampling tube 52 starts at a branch point 34 between the patient-side coupling unit 21 on the one hand and the tube 20 or the Y-piece 22 on the other hand. Therefore, sometimes a gas mixture Gg from the inhalation breathing gas line 32 and sometimes a gas mixture At for the exhalation breathing gas line 33 are fed into the sampling tube 52. Optionally a valve, not shown, is arranged at the branch point 34 which valve in the closed position disconnects the sampling tube 52 from the ventilation circuit 40 and which valve can be controlled by the ventilation control unit 35. When the valve is fully open or omitted, the sampling tube 52 is in an unrestricted fluid connection (fluid communication) with the ventilation circuit 40. The discharge tube 56 leads to a confluence point (entry point) 37 upstream of the carbon dioxide absorber 25a.

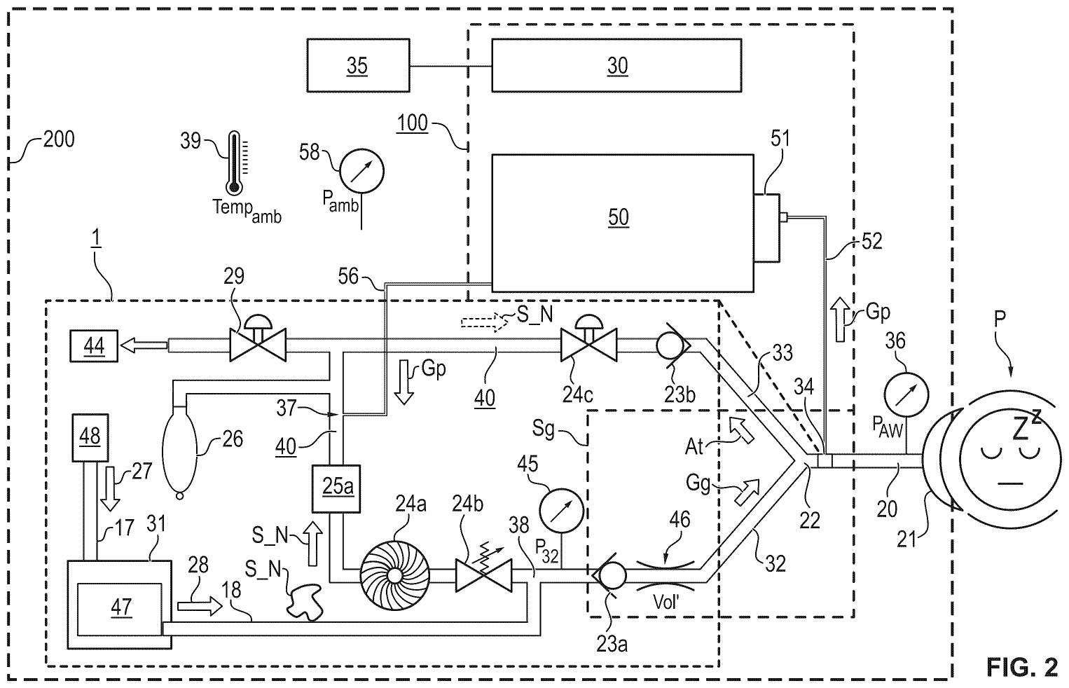

The sampling tube 52 directs the gas sample Gp to a sensor arrangement 50 with its own signal processing unit 30. This sensor arrangement 50 is spatially remote from the patient-side coupling unit 21 and from the breathing gas lines 32 and 33 and also belongs, for example, to the anesthesia machine 1 shown schematically. In FIG. 1 and FIG. 2, the sensor arrangement 50 is shown outside the anesthesia machine 1 for clarity.

In the embodiment example, the sensor arrangement 50 comprises a pump 55, which sucks the gas sample Gp out of the ventilation circuit 40 and sucks it in through the sampling tube 52. A water trap 51 is arranged at the inlet of the sensor arrangement 50, whereby moisture is condensed and collected in the water trap 51. This reduces the water content in the gas sample Gp.

In the embodiment example, the sensor arrangement 50 comprises the following sensors, each of which measures the concentration of a component in the gas sample Gp:
a sensor 59 for the anesthetic concentration (AGas), a sensor 54 for the carbon dioxide concentration (CO₂) and
a sensor 53 for the oxygen concentration (O₂), which preferably utilizes the fact that oxygen is a paramagnetic gas.

The sensor arrangement 50 is configured, for example, as described in DE 10 2021 126 106 A1 (corresponding US application US2023114548 (A1) is incorporated herein by reference).

In addition, the sensor arrangement 50 comprises a pressure sensor 57, which measures the pressure Pcell of the gas sample Gp at the inlet of the sensor arrangement 50. This pressure Pcell is variable over time because the pressure in the ventilation circuit 40 oscillates and because the sampling tube 52 is in a fluid connection with the ventilation circuit 40 if there is no valve at the branch point 34 or as long as the optional valve at the branch point 34 is open. If the valve is omitted or open, the pressure in the ventilation circuit 40 propagates at approximately the sound speed to the sensor arrangement 50.

In the example shown, the sensors 59, 54 and 53 are connected in series and the pressure sensor 57 is arranged in parallel to these three sensors. A different arrangement is also possible.

The sensor arrangement 50 with the pump 55, the water trap 51, the signal processing unit 30 and the tubes (hoses) 52 and 56 belong to a measuring system 100, which extracts the gas sample Gp from the ventilation circuit 40, examines it and feeds it back into the ventilation circuit 40. The measuring system 100 is part of the ventilation arrangement 200.

The breathable gas mixture Gg, which flows through the inhalation breathing gas line 32 and the Y-piece 22 to the patient-side coupling unit 21, is permanently or at least temporarily composed of two different fluid flows, see FIG. 1:
from the fluid flow in the ventilation circuit 40, which has flowed through the exhalation breathing gas line 33 and leaves the carbon dioxide absorber 25a, and
from the fluid flow 28 generated by the anesthetic vaporizer 31, which therefore comprises anesthetic and is fed into the inhalation breathing gas line 32 at the feed point 38.

The following event can occur if no suitable countermeasure is taken: The amount per unit time of anesthetic agent flowing through the inhalation breathing gas line 32 to the patient-side coupling unit 21 is rapidly increased, in particular abruptly increased. One reason for this is the following chain of effects:
A user or a higher-level control system explicitly specifies that the volume flow of the fluid flow 27 should be increased, i.e. that the mixer 48 should cause a larger volume flow. The composition of the fluid flow 28 should remain unchanged.
The mixer 48 is actuated (controlled) in accordance with the specification and causes an increased actual volume flow of carrier gas due to the actuation. This increased volume flow flows through the line 17 as part of the fluid flow 27.
This also increases the volume flow of the fluid flow 28 that leaves the anesthetic vaporizer 31. The increased volume flow leads to a surge S_N through the line 18, see FIG. 2.
The volume flow with which the additional gas mixture is fed at the feed point 38 into the inhalation breathing gas line 32 also increases.
As a result of the greater volume flow, the anesthetic agent in the line 18 from the anesthetic vaporizer 31 to the feed point 38 is fed into the inhalation breathing gas line 32 in a shorter period of time.

For simplicity, it is stated below that the following is effected: The specification that the volume flow of the fluid flow 27 is to be increased results in a surge of carrier gas flowing to the anesthetic vaporizer 31 as a component of the fluid flow 27. One consequence of this is the following: As part of the fluid flow 28, the surge S_N of carrier gas with anesthetic flows from the anesthetic vaporizer 31 to the feed point 38.

Preferably, the anesthetic vaporizer 31 is controlled with the objective of ensuring that the proportion of anesthetic in the fluid flow 28 remains constant. Or the volume flow and/or the mass flow of anesthetic should remain constant. In response to the fact that the volume flow out of the mixer 48 is increased, in many cases the anesthetic vaporizer 31 reduces the proportion of anesthetic in the fluid flow 28 leaving the anesthetic vaporizer 31. Despite this control, it is generally unavoidable that a surge S_N with a relatively high quantity of anesthetic flows through the line 18 after the captured specification has been executed. This is because this reduction inevitably no longer affects the gas mixture that has already left the anesthetic vaporizer 31 before the anesthetic vaporizer 31 reduces the anesthetic content.

The ventilation control unit 35 inevitably detects an increased volume flow Vol′ through the inhalation breathing gas line 32 with a certain time delay, for example due to a signal from the volume flow sensor 46. The anesthetic sensor 59 of the sensor arrangement 50 also inevitably detects a possibly changed anesthetic concentration with a certain time delay. In addition, the ventilation circuit 40 does not necessarily include an outlet in the segment Sg through which excess anesthetic could be discharged.

Without a suitable countermeasure, the following danger exists: The breathable gas mixture Gg, which reaches the patient-side coupling unit 21, contains a too large quantity of anesthetic due to the surge S_N, which flows at the feed point 38 into the ventilation circuit 40. This excessive amount flows through the inhalation breathing gas line 32, reaches the patient-side coupling unit 21 and thus the patient P and can endanger the patient P.

The ventilation strokes should be synchronized with the patient's own respiratory activity. It is therefore generally not a sensible countermeasure to postpone or otherwise change a ventilation stroke.

It would be conceivable to limit the increase in the volume flow of the fluid flow 27. According to this conceivable remedy, the volume flow per unit of time would therefore increase by a predetermined value at most. However, this reduces the flexibility in the use of the ventilation arrangement 200. The invention, on the other hand, provides a way in which the volume flow of the fluid flow 28 can be increased quickly without endangering the patient P.

One objective is therefore to prevent an excessive amount of anesthetic from reaching the patient P at times. Another conceivable remedy would be the following measure: The step of increasing the volume flow out of the mixer 48, i.e. that a surge of carrier gas leaves the mixer 48, triggers the following step: It is prevented that the surge S_N with anesthetic, which is fed in the feed point 38 into the circuit 40, reaches the patient-side coupling unit 21. Instead, at least part of the surge S_N is diverted out of the ventilation circuit 40. For example, an outlet may be provided in segment Sg. In many cases, however, this conceivable remedy results in anesthetic being wasted. Furthermore, this conceivable remedy requires an additional fluid guide unit. The invention provides a way to reduce the risk of endangering the patient P without wasting a lot of anesthetics.

It would also be conceivable to provide a controllable valve instead of a mechanical non-return valve 23a. The invention can be implemented in combination with a controllable valve. However, it is sufficient for the valve 23a to be a mechanically acting non-return valve.

A basic feature of the countermeasure according to the invention is that the following is ensured: The step that the mixer 48 provides an increased volume flow of carrier gas, i.e. that a surge of carrier gas leaves the mixer 48, results in a surge S_N with anesthetic agent being fed at the feed point 38 into the ventilation circuit 40 only in an expiration phase or in an optional intermediate phase, but not in an inspiration phase. By ensuring this, the following is achieved in the example embodiment: In an expiration phase or in an intermediate phase, thanks to the non-return valve 23a, the inhalation breathing gas line 32 is usually interrupted because the patient P exhales, and the injected surge S_N with the larger amount of anesthetic flows from the feed point 38 to the rebreathing system with the carbon dioxide absorber 25a. This situation is illustrated in FIG. 2. Arrows illustrate how the injected surge S_N flows away from the feed point 38, i.e. not into the segment Sg. In the embodiment example, this reverse flow direction is caused by the fact that the surge S_N is fed in with an increased volume flow and therefore generally also with an increased pressure at the feed point 38 and optionally because the non-return valve 23a is closed in an expiration phase and in an intermediate phase.

The injected surge S_N with anesthetic mixes in the ventilation circuit 40 and outside the segment Sg with the exhaled gas mixture At that has left the carbon dioxide absorber 25a, optionally also with exhaled gas mixture At upstream of the carbon dioxide absorber 25a. In the subsequent inspiration phase, the non-return valve 23a is open and the surge S_N with anesthetic flows as part of the breathable gas mixture Gg through the inhalation-breathing gas line 32 to the patient-side coupling unit 21.

Note: Even if the surge S_N leads to the non-return valve 23a being opened in an expiration phase or if the non-return valve 23a is omitted, the gas mixture At exhaled by patient P counteracts this surge S_N in segment Sg. Also in this situation the surge S_N mixes with the gas mixture At exhaled by patient P. As a result, the concentration of anesthetic in the inhalation breathing gas line 32 is generally reduced, even in the case of a large surge S_N.

The ventilation control unit 35 triggers the ventilation strokes and thus causes the inspiration phases. If patient P is not completely anesthetized but is performing his/her own breathing activity, ideally these ventilation strokes are synchronized with the patient's own breathing activity. A ventilation stroke therefore ideally begins and ends with an inhalation process of the patient P. In addition, the ventilation control unit 35 receives a signal from the volume flow sensor 46 and a signal from the pressure sensor 36. The ventilation control unit 35 therefore “knows” when an inspiration phase begins and when it ends and when an expiration phase begins and when it ends. In addition, the ventilation control unit 35 captures a specification that the fluid flow 28 should have a higher target volume flow. To execute this specification, the mixer 48 must increase the volume flow of the fluid flow 27, i.e. expel more carrier gas per unit of time. The captured specification includes the desired target volume flow that the mixer 48 should achieve.

A time period elapses between the following two events:
Due to a corresponding control, the mixer 48 causes the fluid flow 27 with the carrier gas leaving the mixer 48 to have a higher volume flow than before. This generates the surge with carrier gas.
The resulting higher volume flow of the fluid flow 28 with the carrier gas and with the anesthetic (surge S_N) reaches the feed point 38.

The duration of this period depends in particular on the following two parameters:
a volume of a dead space described below and
the volume flow that the mixer 48 produces according to the specification.

The volume of this dead space is the sum of the following volumes:
the volume of the fluid guide unit 18, which guides the fluid flow 28 from the anesthetic vaporizer 31 to the feed point 38,
the volume of the evaporation chamber 47, and
the volume of the fluid guide unit 17, which guides the fluid flow 27 from the mixer 48 to the anesthetic vaporizer 31.

These three volumes are known due to the configuration and design of the ventilation arrangement 200 and generally remain invariable during use. In many cases, it is reasonable to assume that the actual volume flow out of the mixer 48 will match the desired and predetermined volume flow sufficiently closely after the captured specification has been executed. In this case, the duration of the time period is equal to the quotient of the dead space volume and the actual volume flow. It is also possible that a volume flow sensor not shown measures the actual volume flow out of the mixer 48.

The peak of the surge S_N requires this time period to flow from the mixer 48 to the feed point 38. The duration (length, time span) of this period is referred to below as the “propagation duration”.

As already explained, it is ensured that the surge S_N in the fluid flow 28 flowing through the feed line 18 reaches the feed point 38 exclusively during an expiration phase and/or an intermediate phase and that the feed occurs completely outside an inspiration phase. As a rule, the non-return valve 23a is closed during an expiration phase, i.e. when the surge S_N, i.e. the increased volume flow of the fluid flow 28, reaches the feed point 38. The anesthetic in the fluid flow 28 mixes with the exhaled gas mixture At in the ventilation circuit 40, outside the segment Sg and above all in the rebreathing system comprising the carbon dioxide absorber 25a. In the implementation shown, only an expiration phase, but not an intermediate phase, is used to feed the surge S_N into the ventilation circuit 40. According to the invention, it is also possible to use the preceding and/or subsequent intermediate phase for feeding in addition to an expiration phase.

It is important that the non-return valve 23a remains closed long enough after the increased volume flow, i.e. the surge S_N, has been fed in so that sufficient time is available for the mixing just described. The minimum time required for this mixing is referred to below as the “mixing duration”.

As already explained, the invention ensures that the surge S_N is fed into the ventilation circuit 40 at the feed point 38 only in an expiration phase and/or in an intermediate phase. As just explained, preferably at least the mixing duration elapses between the start of the feed and the end of the expiration phase or the end of the subsequent intermediate phase.

As already explained, the ventilation control unit 35 captures a specification that the volume flow out of the mixer 48 should be increased. After capturing this specification, the ventilation control unit 35 predicts the propagation duration Tₚᵣₒₚ and the mixing duration Tₘᵢₓ. The propagation duration Tₚᵣₒₚ depends on the captured required volume flow out of the mixer 48 and the dead space volume. A standard (default) value can be specified for the mixing duration Tₘᵢₓ, which is captured by the ventilation control unit 35. Alternatively, the ventilation control unit 35 derives the mixing duration Tₘᵢₓ from a ventilation frequency and/or at least one other parameter of the artificial ventilation.

Using the propagation duration Tₚᵣₒₚ and the mixing duration Tₘᵢₓ, the ventilation control unit 35 predicts when the start of the surge S_N would reach the feed point 38 if the captured specification were executed immediately, i.e. if the mixer 48 were to immediately effect the required increased volume flow. This predicted time is referred to as tₚᵣₑ. The ventilation control unit 35 checks whether the predicted time tₚᵣₑ falls in an inspiration phase or in an expiration phase or in an intermediate phase. If the predicted time tₚᵣₑ falls in an expiration phase or in an intermediate phase and if there is sufficient time for mixing, the ventilation control unit 35 causes the mixer 48 to effect the increased volume flow now, i.e. without an intended time delay. If, however, the predicted time tₚᵣₑ falls in an inspiration phase and/or there is not enough time for mixing, the ventilation control unit 35 determines a delay time T_{delay}. This delay time T_{delay} is the time interval between the predicted time tₚᵣₑ and the expiration phase, which is the first expiration phase after the predicted time tₚᵣₑ. The ventilation control unit 35 causes the mixer 48 to effect the increased volume flow not immediately, but with a time delay, whereby the duration of the time delay is at least equal to the determined delay duration T_{delay}, optionally greater.

The delay time is determined on the basis of the following boundary conditions (constraints):
The surge S_N requires the propagation duration Tₚᵣₒₚ to flow from the mixer 48 to the feed point 38.
At least the mixing duration Tₘᵢₓ elapses between the time at which the surge S_N reaches the feed point 38 and the end of the expiration phase, in which this reaching time lies.

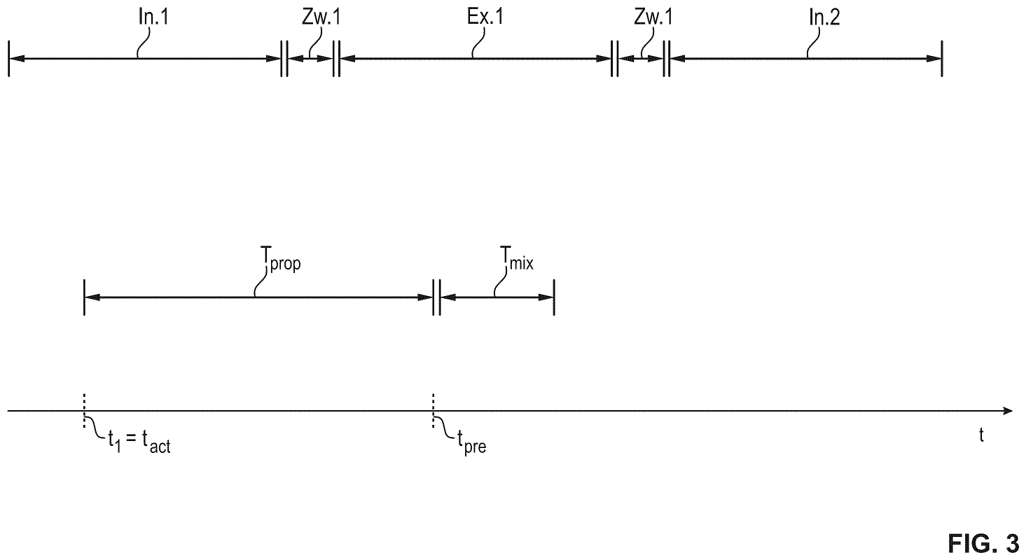

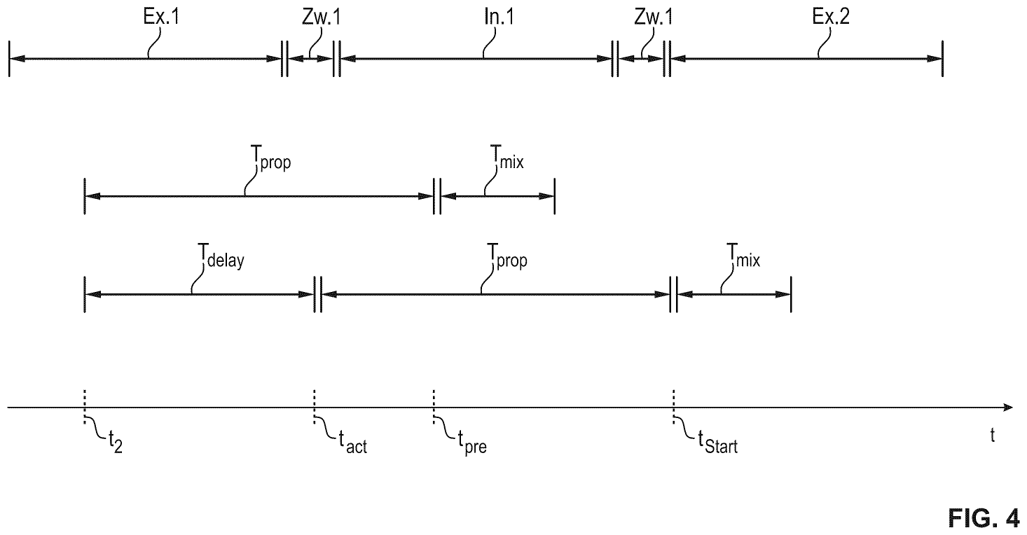

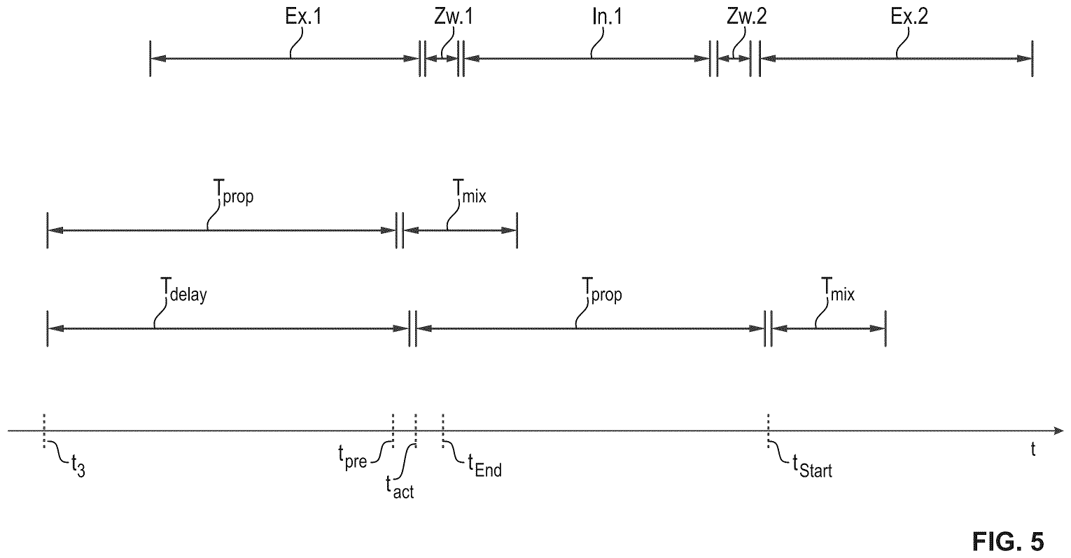

FIG. 3, FIG. 4 and FIG. 5 illustrate the countermeasure according to the invention on a time axis. In.1 and In.2 each denote an inspiration phase, while Ex.1 and Ex.2 each denote an expiration phase. In the embodiment example, an intermediate phase Zw.1, Zw.2 occurs between an inspiration phase and an expiration phase, in which neither a breathable gas mixture Gg flows to the patient-side coupling unit 21 with a volume flow above a predetermined threshold nor an exhaled gas mixture At flows away from the patient-side coupling unit 21. The term tact refers to the time at which the ventilation control unit 35 actually triggers the execution of the captured specification.

In the example in FIG. 3, the ventilation control unit 35 captures at time t₁ the specification that the volume flow from the mixer 48 should be increased. The ventilation control unit 35 predicts the propagation duration Tₚᵣₒₚ and the time tₚᵣₑ. The ventilation control unit 35 determines that the time tₚᵣₑ falls in an expiration phase Ex.1 and that at least the mixing duration Tₘᵢₓ elapses between the time tₚᵣₑ and the end of this expiration phase Ex.1. Therefore, the ventilation control unit 35 causes the specification to be executed immediately, i.e. at time t₁.

In the example in FIG. 4, the ventilation control unit 35 captures at time t₂ the specification that the volume flow from the mixer 48 should be increased. Again, the ventilation control unit 35 predicts the propagation duration Tₚᵣₒₚ and the time tₚᵣₑ. The ventilation control unit 35 detects that the time tₚᵣₑ falls within an inspiration phase In.1. The ventilation control unit 35 determines the time tₛₜₐᵣₜ at which the next expiration phase Ex.2 begins. The ventilation control unit 35 triggers the process of increasing the volume flow from the mixer 48 at time tact, in this case with a delay of T_{delay}. The trigger time tact is set so that the propagation duration Tₚᵣₒₚ just elapses between the trigger time tact and the start tₛₜₐᵣₜ of the next expiration phase Ex.2. The surge S_N therefore reaches the feed point 38 at the start tₛₜₐᵣₜ of the next expiration phase Ex.2. In this expiration phase Ex.2, at least the mixing duration Tₘᵢₓ is available for mixing.

In the example shown in FIG. 5, the ventilation control unit 35 captures the specification at time t₃. The predicted time tₚᵣₑ falls within an expiration phase Ex.1. However, between the predicted time tₚᵣₑ and the end t_{End} of this expiration phase Ex.1, the mixing duration Tₘᵢₓ is not available, but only a shorter time period. Therefore, also in this situation the ventilation control unit 35 delays the step of triggering the execution of the captured specification. The ventilation control unit 35 determines the start tₛₜₐᵣₜ of the next expiration phase Ex.2 and sets the trigger time tact so that the propagation duration Tₚᵣₒₚ lies between the trigger time tact and the start tₛₜₐᵣₜ of the next expiration phase Ex.2.

While specific embodiments of the invention have been shown and described in detail to illustrate the application of the principles of the invention, it will be understood that the invention may be embodied otherwise without departing from such principles.

List of reference characters

| | |
|---|---|
| 1 | Ventilator in the form of an anesthesia machine, performs ventilation |
| | strokes, expels in each ventilation stroke a respective quantity of the |
| | breathable gas mixture Gg |
| 17 | Carrier gas line, leads from the mixer 48 to the anesthetic vaporizer 31, |
| | guides/conducts the fluid flow 27 |
| 18 | Feed line, leads from the anesthetic vaporizer 31 to the feed point 38, |
| | guides/conducts the fluid flow 28 and the surge S_N |
| 20 | Tube in the form of a corrugated tube, connects the patient-side coupling |
| | unit 21 with the Y-piece 22 |
| 21 | Patient-side coupling unit, arranged on and/or in the body of the patient P, |
| | connected to the Y-piece 22 via the hose 20 |
| 22 | Y-piece, connects the two breathing gas lines 32 and 33 to the patient- |
| | side coupling unit 21 via the hose 20 |
| 23a | Non-return valve in the inhalation breathing gas line 32 |
| 23b | Non-return valve in the exhalation breathing gas line 33 |
| 24a | In the embodiment example, the fluid conveying unit that moves the gas |
| | mixture in the ventilation circuit 40 takes the form of a fan that generates |
| | a constant volume flow |
| 24b | Controllable proportional valve, causes the ventilation strokes |
| 24c | PEEP valve, ensures a sufficiently high pressure in the patient's lungs P |
| 25a | Carbon dioxide absorber, removes carbon dioxide from the exhaled gas |
| | mixture At |
| 27 | Fluid flow with a carrier gas, flows in the carrier gas line 17 from the |
| | mixer 48 to the anesthetic vaporizer 31 |
| 28 | Fluid flow with a carrier gas and an anesthetic agent, flows in the feed |
| | line 18 from the anesthetic vaporizer 31 to the feed point 38, contains the |
| | surge S_N |
| 29 | Pressure relief valve in the ventilation circuit 40 |
| 30 | Signal processing unit of the sensor arrangement 50 |
| 31 | Anesthetic vaporizer, generates the fluid flow 28 with vaporous anesthetic |
| | from the fluid flow 27 with a carrier gas and from liquid anesthetic and |
| | feeds this into the feed line 18, comprises the evaporation chamber 47 |
| 32 | Inhalation breathing gas line for inhalation (inspiration), leads from the |
| | carbon dioxide absorber 25a to the Y-piece 22 |
| 33 | Breathing gas line for exhalation (expiration), leads from the Y-piece 22 |
| | to the carbon dioxide absorber 25a |
| 34 | Branch-off point at which the gas sample Gp is branched off from the |
| | ventilation circuit 40 |
| 35 | Ventilation control unit, controls the mixer 48 and the anesthetic |
| | vaporizer 31, processes signals from the sensors 36, 46, 39, 58 |
| 36 | Pressure sensor that measures the airway pressure (pressure in airway, |
| | P_{aw}) on the patient P |
| 37 | Confluence point (entry point) at which the gas sample Gp is fed back |
| | into the ventilation circuit 40 |
| 38 | Feed point at which the fluid flow 28 is fed into the ventilation circuit 40 |
| 39 | Temperature sensor, measures the ambient temperature Temp_{amb} |
| 40 | Ventilation circuit between the anesthesia machine 1 and the patient-side |
| | coupling unit 21, in which a breathable gas mixture Gg is guided to the |
| | patient-side coupling unit 21 and exhaled gas mixture At is guided back |
| | to the anesthesia machine 1 |
| 44 | Conditioner (purifier), extracts breath containing anesthetics from exhaled |
| | gas mixture, anesthetics |
| 45 | Pressure sensor, measures the pressure P₃₂ in the inhalation breathing gas |
| | line 32 |
| 46 | Volume flow sensor, measures the volume flow Vol′ through the |
| | inhalation breathing gas line 32 |
| 47 | Evaporation chamber of the anesthetic vaporizer 31 |
| 48 | Mixer, generates the carrier gas from the components breathing air |
| | and/or pure oxygen (O₂) and optionally nitrous oxide (N₂O) and feeds the |
| | carrier gas into the carrier gas line 17 |
| 50 | Sensor arrangement, analyzes the tapped gas sample Gp, comprises the |
| | sensors 53, 54, 57 and 59, the pump 55 and the control unit 30 |
| 51 | Water trap at the input of the sensor arrangement 50 |
| 52 | Sampling tube (sampling hose) for the gas sample Gp, starts at branch |
| | point 34 and leads to the sensor arrangement 50 |
| 53 | Sensor for the oxygen concentration in the gas sample Gp |
| 54 | Sensor for the carbon dioxide concentration in the gas sample Gp |
| 55 | Pump of the sensor arrangement 50, draws the gas sample Gp from the |
| | ventilation circuit 40 |
| 56 | Discharge tube |
| 57 | Pressure sensor that measures the pressure P_{cell} of the gas sample Gp at |
| | the inlet of the sensor arrangement 50 |
| 58 | Pressure sensor that measures the air pressure P_{amb} in the environment |
| 59 | Sensor for the anesthetic concentration in the gas sample Gp |
| 100 | Measuring system, comprises the sensor arrangement 50, the water trap |
| | 51, the signal processing unit 30 and the tubes 52 and 56 |
| 200 | Ventilation arrangement, comprising the anesthesia machine 1, the |
| | breathing gas lines 32 and 33, the tube 20, the Y-piece 22, the patient-side |
| | coupling unit 21, the measuring system 100, the sensors 36 and 46 and |
| | the optional sensors 39 and 58, provides the ventilation circuit 40 |
| At | Gas mixture exhaled by the patient P into the patient-side coupling unit |
| | 21 and flowing in the ventilation circuit 40 to the anesthesia machine 1 |
| Ex. 1, Ex. 2 | Expiration phase |
| In. 1, In. 2 | Inspiration Phase |
| Gg | Breathable gas mixture, which is expelled from the anesthesia machine 1 |
| | and conducted/guided through the inhalation breathing gas line 32 to the |
| | patient-side coupling unit 21, contains an anesthetic agent and a carrier |
| | gas with oxygen |
| Gp | Gas sample, which is branched off from the ventilation circuit 40 at |
| | branch point 34 and fed back into the ventilation circuit 40 at confluence |
| | point 37 |
| P | Patient, is connected to the patient-side coupling unit 21, is artificially |
| | ventilated and anesthetized |
| P_{amb} | Ambient pressure, measured by pressure sensor 58 |
| P_{aw} | Airway pressure in the patient-side coupling unit 21, measured by the |
| | pressure sensor 36 |
| S_N | Surge of carrier gas with anesthetic agent which, in response to the |
| | volume flow being increased out of mixer 48 reaching the anesthetic |
| | vaporizer 31, flows from the anesthetic vaporizer 31 through the line 18 |
| | to the feed point 38 where it is fed into the ventilation circuit 40 |
| Sg | Segment of the ventilation circuit 40 that is released or blocked by the |
| | non-return valve 23a |
| t₁, t₂, t₃ | Points in time at which the ventilation control unit 35 captures a |
| | specification to increase the volume flow from the mixer 48 |
| t_{act} | Time at which the ventilation control unit 35 triggers the step of |
| | increasing the volume flow from the mixer 48 according to the |
| | specification and thus generating the surge S_N |
| T_{delay} | Delay time that elapses between the capturing of the specification to |
| | increase the volume flow from the mixer 48 and the execution of the |
| | specification, i.e. the generation of the surge S_N |
| t_{End} | End of an expiration phase Ex. 1, Ex. 2 |
| t_{Start} | Start of an expiration phase Ex. 1, Ex. 2 |
| Tₘᵢₓ | Mixing duration required by the surge S_N to mix with exhaled gas |
| | mixture At in an expiration phase Ex. 1, Ex. 2 in the ventilation circuit 40 |
| tₚᵣₑ | Predicted point in time at which the surge S_N would reach the feed point |
| | 38 if the captured specification were to be executed immediately |
| Tₚᵣₒₚ | Propagation duration required by the surge S_N to flow from mixer 48 to |
| | feed point 38 |
| Temp_{amb} | Ambient temperature, measured by temperature sensor 39 |
| Vol′ | Volume flow through the inhalation breathing gas line 32, measured by |
| | the volume flow sensor 46 |
| Zw. 1, Zw. 2 | Intermediate phase between an inspiration phase and an expiration phase |

What is claimed is:

1. A ventilation arrangement for artificial ventilation of a patient that is at least temporarily connected to a patient-side coupling unit, the ventilation arrangement comprising:
a ventilator configured to perform a sequence of ventilation strokes and to expel with each ventilation stroke a respective quantity of a breathable gas mixture comprising oxygen;
a signal-processing control unit; and
a gas mixture supply unit,
wherein the ventilation arrangement provides a ventilation circuit and is configured to guide the respective quantity of breathable gas mixture expelled in each ventilation stroke in the ventilation circuit to the patient-side coupling unit,
wherein the ventilation arrangement is configured to guide a quantity of a gas mixture exhaled by the patient from the patient-side coupling unit in the ventilation circuit to the ventilator,
wherein the gas mixture supply unit is configured to expel an additional gas mixture,
wherein the ventilation arrangement is configured to guide the expelled additional gas mixture to a feed point and at the feed point into the ventilation circuit,
wherein the control unit is configured to capture a specification specifying that the gas mixture supply unit is to expel the additional gas mixture at a target volume flow specified by the specification,
wherein the control unit is configured, in response to capturing the specification, to predict a propagation duration depending on the specified target volume flow, the propagation duration being the time span beginning at a time at which the gas mixture supply unit expels the additional gas mixture in accordance with the captured specification and ending at a time at which that the expelled additional gas mixture reaches the feed point,
wherein the control unit is configured, depending on the predicted propagation duration, to control the gas mixture supply unit such that the controlled gas mixture supply unit expels the additional gas mixture, in accordance with the captured specification, such that the additional gas mixture reaches the feed point within an expiration phase and/or within an intermediate phase,
wherein an expiration phase is a period during which an exhaled gas mixture flows from the patient-side coupling unit to the ventilator, but no breathable gas mixture flows due to a ventilation stroke from the ventilator to the patient-side coupling unit, and
wherein an intermediate phase is a period during which no breathable gas mixture flows due to a ventilation stroke from the ventilator to the patient-side coupling unit and no exhaled gas mixture flows from the patient-side coupling unit to the ventilator.

2. A ventilation arrangement according to claim 1, further comprising a fluid guide unit, which leads from the gas mixture supply unit to the feed point, wherein the control unit is configured to determine the propagation duration depending on a predetermined volume of the fluid guide unit.

3. A ventilation arrangement according to claim 1, wherein a mixing duration is specified and wherein the control unit is configured to control the gas mixture supply unit such that during the expiration phase and/or during the intermediate phase in which the expelled additional gas mixture reaches the feed point at least the mixing duration is available for the expelled additional gas mixture to mix with a gas mixture already present in the ventilation circuit.

4. A ventilation arrangement according to claim 1, further comprising a valve which is arranged in the ventilation circuit between the feed point and the patient-side coupling unit, wherein the ventilation arrangement is configured such that the valve releases a segment in the ventilation circuit between the feed point and the patient-side coupling unit during a ventilation stroke, and blocks the segment in the expiration phase and blocks the segment in the intermediate phase.

5. A ventilation arrangement according to claim 1, wherein the gas mixture supply unit comprises an anesthetic vaporizer adapted to generate a gas mixture comprising a carrier gas and at least one anesthetic agent, and wherein the gas mixture supply unit is configured to expel a gas mixture generated by the anesthetic vaporizer as the additional gas mixture.

6. A ventilation arrangement according to claim 5,
wherein the anesthetic vaporizer comprises an evaporation chamber and the anesthetic vaporizer is configured to feed the or at least one anesthetic of the additional gas mixture into the carrier gas in the evaporation chamber, and
wherein the control unit is configured to additionally predict the propagation duration depending on a predetermined volume of the evaporation chamber.

7. A ventilation arrangement according to claim 1, wherein the target volume flow, that is specified in the captured specification, is at least 50% greater than an actual volume flow, wherein the gas mixture supply unit expels the additional gas mixture with the actual volume flow when the specification is captured.

8. A ventilation process for artificial ventilation of a patient, wherein the patient is at least temporarily connected to a patient-side coupling unit, the process comprising the steps of:
providing a ventilation arrangement, wherein the ventilation arrangement comprises a ventilator and a gas mixture supply unit and provides a ventilation circuit;
with the ventilator, performing a sequence of ventilation strokes and expelling with each ventilation stroke a respective quantity of a breathable gas mixture comprising oxygen,
guiding the respective quantity of breathable gas mixture expelled in one ventilation stroke in the ventilation circuit to the patient-side coupling unit,
guiding a respective quantity of a gas mixture exhaled by the patient from the patient-side coupling unit in the ventilation circuit to the ventilator;
carrying out at least once a sequence, the sequence comprising the steps of expelling an additional gas mixture with the gas mixture supply unit, guiding the expelled additional gas mixture to a feed point, and feeding at the feed point the expelled additional gas mixture into the ventilation circuit; and
prior to carrying out the sequence, capturing a specification specifying that the gas mixture supply unit is to expel the additional gas mixture at a target volume flow specified in the specification, wherein the capturing of this specification triggers the steps of:
depending on the specified target volume flow, predicting a propagation duration, the propagation duration being the time span beginning at a time at which the gas mixture supply unit expels the additional gas mixture in accordance with the captured specification and ending at a time at which the expelled additional gas mixture reaches the feed point; and
depending on the predicted propagation duration, controlling the gas mixture supply unit so as to expel the additional gas mixture in accordance with the captured specification such that the additional gas mixture reaches the feed point within an expiration phase and/or within an intermediate phase, wherein an expiration phase is a period during which an exhaled gas mixture flows from the patient-side coupling unit to the ventilator, but no breathable gas mixture flows due to a ventilation stroke from the ventilator to the patient-side coupling unit, and wherein an intermediate phase is a period during which no breathable gas mixture flows due to a ventilation stroke from the ventilator to the patient-side coupling unit and no exhaled gas mixture flows from the patient-side coupling unit to the ventilator.
